(19) United States
(12) Patent Application Publication
(10) Pub. No.: US 2024/0100160 A1
(43) Pub. Date: Mar. 28, 2024

(54) ENHANCEMENT OF T CELL HOMING TO TUMORS THROUGH AUGMENTATION OF CHEMOKINE RESPONSIVENESS AND ACTIVATION DEPENDENT CHEMOKINE SECRETION

(71) Applicant: Regen Biopharma, Inc., La Mesa, CA (US)

(72) Inventors: Thomas ICHIM, San Diego, CA (US); Harry LANDER, San Diego, CA (US); David KOOS, La Mesa, CA (US)

(73) Assignee: Regen Biopharma, Inc., La Mesa, CA (US)

(21) Appl. No.: 18/475,042

(22) Filed: Sep. 26, 2023

Related U.S. Application Data
(60) Provisional application No. 63/410,205, filed on Sep. 26, 2022.

(51) Int. Cl. A61K 39/00; C07K 14/725; C12N 5/0783

(52) U.S. Cl. CPC A61K 39/4632; A61K 39/4611; C07K 14/7051; C12N 5/0636; A61K 2239/28; C12N 2510/00

(57) ABSTRACT

Disclosed are means, methods and compositions of matter useful for stimulation enhancement of T cell homing into tumors and overcoming tumor microenvironment. In one embodiment the invention provides an engineered T cell in which engagement of T cell receptor on said T cell results in upregulation of T cell attracting chemokines so as to induce an increased proportion of T cells to tumor cells. In another embodiment, T cell chemokine secreting cells are administrated intratumorally in order to augment T cell infiltration into said tumors. In other embodiments administration of lymphopoietic cytokines is performed intratumorally to enhance viability of T cells approaching and residing in the microenvironment. Combinations of agents which counteract tumor microenvironment induced immune suppression are also disclosed.

### CROSS-REFERENCE TO RELATED APPLICATIONS

This application claims priority to U.S. Provisional Application Ser. No. 63/410,205, titled “Enhancement of T Cell Homing to Tumors Through Augmentation of Chemokine Responsiveness and Activation Dependent Chemokine Secretion”, filed Sep. 26, 2022, which is incorporated by reference herein in its entirety.

### FIELD OF THE INVENTION

The teachings herein relate to methods of treating cancer through enhancing CAR-T cells to induce recruitment of other immune cells upon activation of the CAR.

### SUMMARY

Preferred embodiments are directed to methods of treating cancer comprising the steps of: a) obtaining a T cell population possessing a T cell receptor (TCR) capable of recognizing one or more tumor antigens; b) transfecting said T cell population with a construct in a manner such that activation of T cell receptor or signaling components associated with said T cell receptor results in stimulation of chemokine production from said T cell; and c) administering said T cell to a patient in need of treatment.

Preferred methods include embodiments wherein said T cell population is selected from a group of T cells comprising of: a) CD4 T cells; b) CD8 T cells; c) NKT cells; d) gamma delta T cells and e) innate lymphoid like cells.

Preferred methods include embodiments wherein said CD4 cell is selected from a group of cells comprising: a) Th1; b) Th2; c) Th3; d) Th9; and e) Th17.

Preferred methods include embodiments wherein said T cell population is capable of inducing chemoattraction of dendritic cells.

Preferred methods include embodiments wherein said dendritic cell is capable of inducing antigen presentation.

Preferred methods include embodiments wherein said antigen presentation involves expression of MHC I and/or MHCII on the surface of a dendritic cell, in which said MHC I and/or MHC II contains antigenic peptides.

Preferred methods include embodiments wherein said antigen presentation involves expression of costimulatory molecules.

Preferred methods include embodiments wherein said costimulatory molecule is CD40.

Preferred methods include embodiments wherein said costimulatory molecule is CD80.

Preferred methods include embodiments wherein said costimulatory molecule is CD86.

Preferred methods include embodiments wherein said costimulatory molecule is interleukin-15.

Preferred methods include embodiments wherein said costimulatory molecule is interleukin-12.

Preferred methods include embodiments wherein said costimulatory molecule is ICAM-1.

Preferred methods include embodiments wherein said costimulatory molecule is LFA-1.

Preferred methods include embodiments wherein said costimulatory molecule is ICOS ligand.

Preferred methods include embodiments wherein said costimulatory molecule is OX40 ligand.

Preferred methods include embodiments wherein said costimulatory molecule is CD40.

Preferred methods include embodiments wherein said cancer antigen is selected from a group comprising of: Antigens known to be found on cancer, and useful for the practice of the invention include: pidermal growth factor receptor (EGFR, EGFR1, ErbB-1, HER1). ErbB-2 (HER2/neu), ErbB-3/HER3, ErbB-4/HER4, EGFR ligand family; insulin-like growth factor receptor (IGFR) family, IGF-binding proteins (IGFBPs), IGFR ligand family (IGF-1R); platelet derived growth factor receptor (PDGFR) family, PDGFR ligand family; fibroblast growth factor receptor (FGFR) family, FGFR ligand family, vascular endothelial growth factor receptor (VEGFR) family, VEGF family; HGF receptor family: TRK receptor family; ephrin (EPH) receptor family: AXL receptor family; leukocyte tyrosine kinase (LTK) receptor family; TIE receptor family, angiopoietin 1, 2; receptor tyrosine kinase-like orphan receptor (ROR) receptor family; discoidin domain receptor (DDR) family; RET receptor family; KLG receptor family; RYK receptor family; MuSK receptor family; Transforming growth factor alpha (TGF-.alpha.), TGF-.alpha. receptor; Transforming growth factor-beta (TGF-.beta.), TGF-.beta. receptor; Interleukin .beta. receptor alpha2 chain (IL13Ralpha2), Interleukin-6 (IL-6), IL-6 receptor, interleukin-4, IL-4 receptor, Cytokine receptors, Class I (hematopoietin family) and Class II (interferon/1L-10 family) receptors, tumor necrosis factor (TNF) family, TNF-.alpha., tumor necrosis factor (TNF) receptor superfamily (TNTRSF), death receptor family, TRAIL-receptor; cancer-testis (CT) antigens, lineage-specific antigens, differentiation antigens, alpha-actinin-4, ARTC1, breakpoint cluster region-Abelson (Bcr-abl) fusion products, B-RAF, caspase-5 (CASP-5), caspase-8 (CASP-8), beta-catenin (CTNNB1), cell division cycle 27 (CDC27), cyclin-dependent kinase 4 (CDK4), CDKN2A, COA-1, dek-can fusion protein, EFTUD-2, Elongation factor 2 (ELF2), Ets variant gene 6/acute myeloid leukemia 1 gene ETS (ETC6-AML1) fusion protein, fibronectin (FN), GPNMB, low density lipid receptor/GDP-L fucose: beta-Dgalactose 2-alpha-Lfucosyltraosferase (LDLR/FUT) fusion protein, HLA-A2, MLA-A11, heat shock protein 70-2 mutated (HSP70-2M), KIAA0205, MART2, melanoma ubiquitous mutated 1, 2, 3 (MUM-1, 2, 3), prostatic acid phosphatase (PAP), neo-PAP, Myosin class 1, NFYC, OGT, OS-9, pml-RARalpha fusion protein, PRDX5, PTPRK, K-ras (KRAS2), N-ras (NRAS), HRAS, RBAF600, SIRT12, SNRPD1, SYT-SSX1 or -SSX2 fusion protein, Triosephosphate Isomerase, BAGE, BAGE-1, BAGE-2, 3, 4, 5, GAGE-1, 2, 3, 4, 5, 6, 7, 8, GnT-V (aberrant N-acetyl glucosaminyl transferase V, MGAT5), HERV-K MEL, KK-LC, KM-HN-1, LAGE, LAGE-1, CTL-recognized antigen on melanoma (CAMEL), MAGE-A1 (MAGE-1). MAGE-A2, MAGE-A3, MAGE-A4, MAGE-AS, MAGE-A6, MAGE-A8, MAGE-A9, MAGE-A10. MAGE-A11, MAGE-A12, MAGE-3, MAGE-B1, MAGE-B2, MAGE-B5. MAGE-B6, MAGE-C1, MAGE-C2, mucin 1 (MUC1), MART-1/Melan-A (MLANA), gp100, gp100/Pme117 (S1LV), tyrosinase (TYR), TRP-1, HAGE, NA-88, NY-ESO-1, NY-ESO-1/LAGE-2, SAGE, Sp17. SSX-1, 2, 3, 4, TRP2-1NT2, carcino-embryonic antigen (CEA), Kallikrein 4, mammaglobin-A, OA1, prostate specific antigen (PSA), prostate specific membrane antigen, TRP-1/, 75. TRP-2 adipophilin, interferon inducible protein absent in melanoma 2 (AIM-2). BING-4, CPSF, cyclin D1, epithelial cell adhesion molecule (Ep-CAM), EpbA3, fibroblast growth factor-5 (FGF-5), glycoprotein 250 (gp250intestinal carboxyl esterase (iCE), alpha-feto protein (AFP), M-CSF, mdm-2, MUCI, p53 (TP53), PBF, PRAME, PSMA, RAGE-1, RNF43, RU2AS, SOX10, STEAP1, survivin (BIRCS), human telomerase reverse transcriptase (hTERT), telomerase, Wilms' tumor gene (WT1), SYCP1, BRDT, SPANX, XAGE, ADAM2, PAGE-5, LIP1, CTAGE-1, CSAGE, MMA1, CAGE, BORIS, HOM-TES-85, AF15q14, HCA66I, LDHC, MORC, SGY-1, SPO11, TPX1, NY-SAR-35, FTHLI7, NXF2 TDRD1, TEX 15, FATE, TPTE, immunoglobulin idiotypes, Bence-Jones protein, estrogen receptors (ER), androgen receptors (AR), CD40, CD30, CD20, CD19, CD33, CD4, CD25, CD3, cancer antigen 72-4 (CA 72-4), cancer antigen 15-3 (CA 15-3), cancer antigen 27-29 (CA 27-29), cancer antigen 125 (CA 125), cancer antigen 19-9 (CA 19-9), beta-human chorionic gonadotropin, 1-2 microglobulin, squamous cell carcinoma antigen, neuron-specific enolase, heat shock protein gp96. GM2, sargramostim, CTLA-4, 707 alanine proline (707-AP), adenocarcinoma antigen recognized by T cells 4 (ART-4), carcinoembryogenic antigen peptide-1 (CAP-1), calcium-activated chloride channel-2 (CLCA2), cyclophilin B (Cyp-B), and human signet ring tumor-2 (HST-2).

Preferred methods include embodiments wherein said T cells are transfected with a construct that upon activation induces production of CXCL10.

Preferred methods include embodiments wherein said T cells are transfected with a construct that upon activation induces production of CXCL11.

Preferred methods include embodiments wherein upon activation of said CAR said T cell produces a cytokine associated with induction of T cell mediated cancer immunity.

Preferred methods include embodiments wherein said cytokine associated with induction of said T cell mediated cancer immunity is interferon alpha.

Preferred methods include embodiments wherein said cytokine associated with induction of said T cell mediated cancer immunity is interferon beta.

Preferred methods include embodiments wherein said cytokine associated with induction of said T cell mediated cancer immunity is interferon gamma.

Preferred methods include embodiments wherein said cytokine associated with induction of said T cell mediated cancer immunity is interferon tau.

Preferred methods include embodiments wherein said cytokine associated with induction of said T cell mediated cancer immunity is interferon omega.

Preferred methods include embodiments wherein said cytokine associated with induction of said T cell mediated cancer immunity is interleukin-1.

Preferred methods include embodiments wherein said cytokine associated with induction of said T cell mediated cancer immunity is interleukin-2.

Preferred methods include embodiments wherein said cytokine associated with induction of said T cell mediated cancer immunity is interleukin-6.

Preferred methods include embodiments wherein said cytokine associated with induction of said T cell mediated cancer immunity is interleukin-7.

Preferred methods include embodiments wherein said cytokine associated with induction of said T cell mediated cancer immunity is interleukin-8.

Preferred methods include embodiments wherein said cytokine associated with induction of said T cell mediated cancer immunity is interleukin-9.

Preferred methods include embodiments wherein said cytokine associated with induction of said T cell mediated cancer immunity is interleukin-11.

Preferred methods include embodiments wherein said cytokine associated with induction of said T cell mediated cancer immunity is interleukin-12.

Preferred methods include embodiments wherein said cytokine associated with induction of said T cell mediated cancer immunity is interleukin-15.

Preferred methods include embodiments wherein said cytokine associated with induction of said T cell mediated cancer immunity is interleukin-17.

Preferred methods include embodiments wherein said cytokine associated with induction of said T cell mediated cancer immunity is interleukin-18.

Preferred methods include embodiments wherein said cytokine associated with induction of said T cell mediated cancer immunity is interleukin-21.

Preferred methods include embodiments wherein said cytokine associated with induction of said T cell mediated cancer immunity is interleukin-23.

Preferred methods include embodiments wherein said cytokine associated with induction of said T cell mediated cancer immunity is interleukin-27.

Preferred methods include embodiments wherein said cytokine associated with induction of said T cell mediated cancer immunity is interleukin-33.

Preferred methods include embodiments wherein said cytokine associated with induction of said T cell mediated cancer immunity is hmgb1.

Preferred methods include embodiments wherein said cytokine associated with induction of said T cell mediated cancer immunity is TRIAL.

Preferred methods include embodiments wherein said cytokine associated with induction of said T cell mediated cancer immunity is BlyS.

Preferred methods include embodiments wherein said cytokine associated with induction of said T cell mediated cancer immunity is interleukin-LIGHT.

Preferred methods include embodiments wherein said cytokine associated with induction of said T cell mediated cancer immunity is RANKL.

Preferred methods include embodiments wherein said cytokine associated with induction of said T cell mediated cancer immunity is osteopontin.

Preferred methods include embodiments wherein said cytokine associated with induction of said T cell mediated cancer immunity is TNF-alpha.

Preferred methods include embodiments wherein said cytokine associated with induction of said T cell mediated cancer immunity is lymphotoxin.

Preferred methods include embodiments wherein said cytokine associated with induction of said T cell mediated cancer immunity is antibody to interleukin-1 receptor antagonist.

Preferred methods include embodiments wherein said cytokine associated with induction of said T cell mediated cancer immunity is antibody to interleukin-3.

Preferred methods include embodiments wherein said cytokine associated with induction of said T cell mediated cancer immunity is antibody to interleukin-4.

Preferred methods include embodiments wherein said cytokine associated with induction of said T cell mediated cancer immunity is antibody to interleukin-5.

Preferred methods include embodiments wherein said cytokine associated with induction of said T cell mediated cancer immunity is antibody to interleukin-10.

Preferred methods include embodiments wherein said cytokine associated with induction of said T cell mediated cancer immunity is antibody to interleukin-13.

Preferred methods include embodiments wherein said cytokine associated with induction of said T cell mediated cancer immunity is antibody to interleukin-16.

Preferred methods include embodiments wherein said cytokine associated with induction of said T cell mediated cancer immunity is antibody to interleukin-20.

Preferred methods include embodiments wherein said cytokine associated with induction of said T cell mediated cancer immunity is antibody to soluble TNF-alpha receptor p55.

Preferred methods include embodiments wherein said cytokine associated with induction of said T cell mediated cancer immunity is antibody to soluble TNF-alpha receptor p75.

Preferred methods include embodiments wherein said cytokine associated with induction of said T cell mediated cancer immunity is antibody to soluble HLA-G.

Preferred methods include embodiments wherein said cytokine associated with induction of said T cell mediated cancer immunity is antibody to TGF-beta.

Preferred methods include embodiments wherein said cytokine associated with induction of said T cell mediated cancer immunity is antibody to ILT-3.

Preferred methods include embodiments wherein said cytokine associated with induction of said T cell mediated cancer immunity is antibody to soluble ILT-4.

Preferred methods include embodiments wherein said cytokine associated with induction of said T cell mediated cancer immunity is antibody to CD47.

Preferred methods include embodiments wherein said cytokine associated with induction of said T cell mediated cancer immunity is antibody to PD-1.

Preferred methods include embodiments wherein said cytokine associated with induction of said T cell mediated cancer immunity is antibody to soluble PD-1 ligand.

Preferred methods include embodiments wherein said cytokine associated with induction of said T cell mediated cancer immunity is antibody to soluble VEGF.

Preferred methods include embodiments wherein said cytokine associated with induction of said T cell mediated cancer immunity is antibody to soluble PDGF.

Preferred methods include embodiments wherein said cytokine associated with induction of said T cell mediated cancer immunity is antibody to soluble FGF-1.

Preferred methods include embodiments wherein said cytokine associated with induction of said T cell mediated cancer immunity is antibody to soluble ILT-4.

Preferred methods include embodiments wherein upon activation of said CAR said T cell produces a cytokine associated with induction of macrophage mediated cancer immunity.

Preferred methods include embodiments wherein said cytokine that is associated with induction of macrophage mediated cancer immunity is interferon gamma.

Preferred methods include embodiments wherein said cytokine that is associated with induction of macrophage mediated cancer immunity is TNF-alpha.

Preferred methods include embodiments wherein said cytokine that is associated with induction of macrophage mediated cancer immunity is interleukin-12.

Preferred methods include embodiments wherein said cytokine that is associated with induction of macrophage mediated cancer immunity is interleukin-15.

Preferred methods include embodiments wherein said cytokine that is associated with induction of macrophage mediated cancer immunity is TNF-alpha.

Preferred methods include embodiments wherein said cytokine that is associated with induction of macrophage mediated cancer immunity is TRAIL.

Preferred methods include embodiments wherein said cytokine that is associated with induction of macrophage mediated cancer immunity is GC-MAF.

Preferred methods include embodiments wherein said cytokine that is associated with induction of macrophage mediated cancer immunity is LIGHT.

Preferred methods include embodiments wherein said cytokine that is associated with induction of macrophage mediated cancer immunity is RANK ligand.

Preferred methods include embodiments wherein said cytokine that is associated with induction of macrophage mediated cancer immunity is M-CSF.

Preferred methods include embodiments wherein said cytokine that is associated with induction of macrophage mediated cancer immunity is GM-CSF.

Preferred methods include embodiments wherein said cytokine that is associated with induction of macrophage mediated cancer immunity is flt-3 ligand.

Preferred methods include embodiments wherein an immunological adjuvant is added to the tumor before administration of CAR-T cells.

Preferred methods include embodiments wherein said immunological adjuvant is administration of dendritic cells locally into the tumor.

Preferred methods include embodiments wherein said dendritic cells are derived from the bone marrow.

Preferred methods include embodiments wherein said dendritic cells are derived from peripheral blood.

Preferred methods include embodiments wherein said dendritic cells are derived from cord blood.

Preferred methods include embodiments wherein said dendritic cells are derived from mobilized peripheral blood.

Preferred methods include embodiments wherein said peripheral blood is mobilized by treatment of the patient with one or more agents capable of increasing the numbers of dendritic cells in circulation.

Preferred methods include embodiments wherein said agent that increases the dendritic cells in circulation is G-CSF.

Preferred methods include embodiments wherein said agent that increases the dendritic cells in circulation is GM-CSF.

Preferred methods include embodiments wherein said agent that increases the dendritic cells in circulation is M-CSF.

Preferred methods include embodiments wherein said agent that increases the dendritic cells in circulation is Flt3-ligand.

Preferred methods include embodiments wherein said agent that increases the dendritic cells in circulation is mozibil.

Preferred methods include embodiments wherein said agent that increases the dendritic cells in circulation is TPO.

Preferred methods include embodiments wherein said agent that increases the dendritic cells in circulation is interleukin-3

Preferred methods include embodiments wherein said agent that increases the dendritic cells in circulation is TNF-alpha.

Preferred methods include embodiments wherein said agent that increases the dendritic cells in circulation is interferon-gamma.

Preferred methods include embodiments wherein said agent that increases the dendritic cells in circulation is interferon alpha.

Preferred methods include embodiments wherein said dendritic cells are generated by treatment of dendritic progenitors with GM-CSF.

Preferred methods include embodiments wherein said dendritic cells are generated by treatment of dendritic progenitors with GM-CSF and interleukin-4.

Preferred methods include embodiments wherein said dendritic cells express CD80.

Preferred methods include embodiments wherein said dendritic cells express CD86.

Preferred methods include embodiments wherein said dendritic cells express CD40.

Preferred methods include embodiments wherein said dendritic cells express IL-12.

Preferred methods include embodiments wherein said dendritic cells express IL-15.

Preferred methods include embodiments wherein said dendritic cells express IL-17.

Preferred methods include embodiments wherein said dendritic cells express IL-18.

Preferred methods include embodiments wherein said dendritic cells express IL-33.

Preferred methods include embodiments wherein said immunological adjuvant activates an immune receptor.

Preferred methods include embodiments wherein said immune receptor is an activator of immunotyrosine activation motifs.

Preferred methods include embodiments wherein said immune receptor is an activator of TNF-alpha signalling

Preferred methods include embodiments wherein said immune receptor activates NF-AT.

Preferred methods include embodiments wherein said immune receptor activates NF-kappa B.

Preferred methods include embodiments wherein said immune receptor activates STAT-3.

Preferred methods include embodiments wherein said immune receptor activates janus activated kinase.

Preferred methods include embodiments wherein said immune receptor activates MAP-kinase.

Preferred methods include embodiments wherein said immune receptor is TLR. 1

Preferred methods include embodiments wherein said TLR-1 is activated by Pam3 CS K4.

Preferred methods include embodiments wherein said immune receptor is TLR-2

Preferred methods include embodiments wherein said TLR-2 is activated by HKLM.

Preferred methods include embodiments wherein said immune receptor is TLR-3.

Preferred methods include embodiments wherein said TLR-3 is activated by Poly: IC.

Preferred methods include embodiments wherein said immune receptor is TLR-4.

Preferred methods include embodiments wherein said TLR-4 is activated by LPS.

Preferred methods include embodiments wherein said TLR-4 is activated by Buprenorphine.

Preferred methods include embodiments wherein said TLR-4 is activated by Carbamazepine.

Preferred methods include embodiments wherein said TLR-4 is activated by Fentanyl.

Preferred methods include embodiments wherein said TLR-4 is activated by Levorphanol.

Preferred methods include embodiments wherein said TLR-4 is activated by Methadone.

Preferred methods include embodiments wherein said TLR-4 is activated by Cocaine.

Preferred methods include embodiments wherein said TLR-4 is activated by Morphine.

Preferred methods include embodiments wherein said TLR-4 is activated by Oxcarbazepine.

Preferred methods include embodiments wherein said TLR-4 is activated by Oxycodone.

Preferred methods include embodiments wherein said TLR-4 is activated by Pethidine.

Preferred methods include embodiments wherein said TLR-4 is activated by Glucuronoxylomannan from Cryptococcus.

Preferred methods include embodiments wherein said TLR-4 is activated by Morphine-3-glucuronide.

Preferred methods include embodiments wherein said TLR-4 is activated by lipoteichoic acid.

Preferred methods include embodiments wherein said TLR-4 is activated by beta.-defensin 2.

Preferred methods include embodiments wherein said TLR-4 is activated by low molecular weight hyaluronic acid.

Preferred methods include embodiments wherein said low molecular weight hyaluronic acid has a molecular weight of <1000 kDa.

Preferred methods include embodiments wherein said low molecular weight hyaluronic acid has a molecular weight of <500 kDa.

Preferred methods include embodiments wherein said low molecular weight hyaluronic acid has a molecular weight of <250 kDa.

Preferred methods include embodiments wherein said low molecular weight hyaluronic acid has a molecular weight of <100 kDa.

Preferred methods include embodiments wherein said TLR-4 is activated by fibronectin EDA.

Preferred methods include embodiments wherein said TLR-4 is activated by snapin.

Preferred methods include embodiments wherein said TLR-4 is activated by tenascin C.

Preferred methods include embodiments wherein said immune receptor is TLR-5.

Preferred methods include embodiments wherein said TLR-5 is activated by flaggelin.

Preferred methods include embodiments wherein said immune receptor is TLR-6.

Preferred methods include embodiments wherein said TLR-6 is activated by FSL-1.

Preferred methods include embodiments wherein said immune receptor is TLR-7.

Preferred methods include embodiments wherein said TLR-7 is activated by imiquimod.

Preferred methods include embodiments wherein said immune receptor is TLR-8.

Preferred methods include embodiments wherein said TLR-8 is activated by ssRNA40/LyoVec.

Preferred methods include embodiments wherein said immune receptor is TLR-9.

Preferred methods include embodiments wherein said TLR-9 is activated by a CpG oligonucleotide.

Preferred methods include embodiments wherein said TLR-9 is activated by ODN2006.

Preferred methods include embodiments wherein said TLR-9 is activated by Agatolimod.

Preferred methods include embodiments wherein said TLR-9 is activated by ODN2007.

Preferred methods include embodiments wherein said TLR-9 is activated by ODN1668.

Preferred methods include embodiments wherein said TLR-9 is activated by ODN1826.

Preferred methods include embodiments wherein said TLR-9 is activated by ODN BW006.

Preferred methods include embodiments wherein said TLR-9 is activated by ODN D SL01.

Preferred methods include embodiments wherein said TLR-9 is activated by ODN 2395.

Preferred methods include embodiments wherein said TLR-9 is activated by ODN M362.

Preferred methods include embodiments wherein said TLR-9 is activated by ODN SL03.

Preferred methods include embodiments wherein said activator of said immune receptor is Fas.

Preferred methods include embodiments wherein said activator of said immune receptor is Trail receptor.

Preferred methods include embodiments wherein said activator of said immune receptor is TNF-alpha receptor p55.

Preferred methods include embodiments wherein said activator of said immune receptor is TNF-alpha receptor p75.

### BACKGROUND OF THE INVENTION

Immunotherapy offers the promise of selectively killing cancer without the side effects of classical cancer therapies such as chemotherapy. Recent approaches aimed at helping the immune system specifically to recognize tumor-specific antigens involve immunization with cancer-specific antigens, typically combined with an adjuvant (a substance which is known to cause or enhance an immune response) to the subject. Tumor specific antigens are well known and include It is known that the usual lack of a powerful immune response to tumor associated antigens (TAAs) is due to a combination of factors. T cells have a key role in the immune response, which is mediated through antigen recognition by the T cell receptor (TCR), and they coordinate a balance between co-stimulatory and inhibitory signals known as immune. Inhibitory signals suppress the immune system which is important for maintenance of self-tolerance and to protect tissues from damage when the immune system is responding to pathogenic infection. However, immune suppression reduces what could otherwise be a helpful response by the body to the development of tumours. Cytokines, other stimulatory molecules such as CpG (stimulating dendritic cells), Toll-like receptor ligands and other molecular adjuvants enhance the immune response. Co-stimulatory interactions involving T cells directly can be enhanced using agonistic antibodies to receptors including OX40, CD28, CD27 and CD137. Other immune system activating therapies include blocking and/or depleting inhibitory cells or molecules and include the use of antagonistic antibodies against what are known as immune checkpoints [41]. It is known that immune cells express proteins that are immune checkpoints that control and down-regulate the immune response. These are best defined in T lymphocytes and include PD-1, CTLA-4, TIM-3 and LAG3. Tumor cells express the ligands to these receptors. When T cells bind the ligand to these proteins on the tumor cells, the T cell is turned off and does not attempt to attack the tumor cell. Thus, checkpoint inhibitors are part of the complex strategy used by the tumor to evade the patient's immune system and are responsible for resistance to immunotherapy. Biopharmaceutical companies have successfully developed checkpoint inhibitors that block the receptor/ligand interaction to promote the adaptive immune response to the tumor. Six checkpoint inhibitors are currently approved, pembrolizumab, nivolumab, atezolizumab, avelumab, durvalumab, and ipilimumab for a wide variety of solid tumors including melanoma, lung, bladder, gastric cancers and others. T cells are central to the immune response to cancers and there is interest in the field in using tumor infiltrating lymphocytes (TILs) in the treatment and understanding of cancer. Through their T cell receptors (TCRs), T cells are reactive to specific antigens within a tumor. Tumor cells carry genetic mutations, many of which contribute directly or indirectly to malignancy. A mutation in an expressed sequence will typically result in a neoantigen, an antigen that is not known to the immune system and thus recognized as foreign and able to elicit an immune response.

Unfortunately, despite the great advances in understanding of the immune-cancer interaction, and development of novel first in class drugs around this concept, many patients still do not respond to immunotherapies, and in some cases, those that respond suffer from relapse. The current invention teaches means of augmenting efficacy of immunotherapies, specifically of chimeric antigen receptor (CAR)-T cells, by mean intra alia, of transfection with inducible expression of chemokines in order to attract other immune cells and therefore overcome tumor immune suppressive microenvironment, which previously has been noted to act as a significant impediment to treatment efficacy of immunotherapy.

### DETAILED DESCRIPTION OF THE INVENTION

The invention provides means and compositions of matter for treatment of cancer through potentiating CAR-T cells to induce recruitment of other immune cells upon activation of the CAR. In some embodiments the CAR-T cell is engineered in a manner to induce expression of T cell attracting chemokines such as CCL7. In other embodiments said CAR-T cells are engineered to secrete activators of innate immunity such as TNF-alpha upon activation of said CAR-T. In some embodiments said TNF-alpha is engineered in a manner to be capable of inducing vascular necrosis so as to cause killing of tumors through starvation of nutrients. In other embodiments said CAR-T cells are engineered to secrete complement components in order to enhance immunogenicity.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

The term “about” is used to indicate that a value includes the inherent variation of error for the device, the method being employed to determine the value, or the variation that exists among the study subjects.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. Other specific types of cancer include cinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti, The term “sarcoma” generally refers to a tumor which is made up of a substance like the embryonic connective tissue and is generally composed of closely packed cells embedded in a fibrillar, heterogeneous, or homogeneous substance. Sarcomas include, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma. Additional exemplary neoplasias include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

In some particular embodiments of the invention, the cancer treated is a melanoma. The term “melanoma” is taken to mean a tumor arising from the melanocytic system of the skin and other organs. Melanomas include, for example, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

The term “chimeric antigen receptors (CARs),” as used herein, may refer to artificial T-cell receptors, chimeric T-cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a tumor associated antigen, for example. In some embodiments, CARs comprise an intracellular activation domain, a transmembrane domain, and an extracellular domain comprising a tumor associated antigen binding region. In particular aspects, CARs comprise fusions of single-chain variable fragments (scFv) derived from monoclonal antibodies, fused to CD3-zeta a transmembrane domain and endodomain. The specificity of other CAR designs may be derived from ligands of receptors (e.g., peptides) or from pattern-recognition receptors, such as Dectins. In certain cases, the spacing of the antigen-recognition domain can be modified to reduce activation-induced cell death. In certain cases, CARs comprise domains for additional co-stimulatory signaling, such as CD3.zeta., FcR, CD27, CD28, CD137, DAP10, DAP12 and/or OX40. In some cases, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

The term “Costimulatory ligand” or “costimulatory molecule” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand can include, but is not limited to, CD7, B7-1 (CD80), B7-2 (CD86), programmed death (PD) L1, PD-L2, 4-1BB ligand, OX40 ligand, inducible costimulatory ligand (ICOS-L), intercellular adhesion molecule (ICAM), CD30 ligand, CD40, CD70, CD83, human leukocyte antigen G (HLA-G), MHC class I chain-related protein A (MICA), MHC class I chain-related protein B (MICB), herpes virus entry mediator (HVEM), lymphotoxin beta receptor, 3/TR6, immunoglobulin-like transcript (ILT) 3, ILT4, an agonist or antibody that binds Toll ligand receptor and a ligand that specifically binds with B7-H3. A co-stimulatory ligand includes, without limitation, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, tumor necrosis factor superfamily member 14 (TNFSF14 or LIGHT), natural killer cell receptor C (NKG2C), B7-H3, and a ligand that specifically binds with CD83.

The term “cytokine”, as used herein, refers to a non-antibody protein that is released by one cell in response to contact with a specific antigen, wherein the cytokine interacts with a second cell to mediate a response in the second cell. A cytokine can be endogenously expressed by a cell or administered to a subject. Cytokines may be released by immune cells, including macrophages, B cells, T cells, and mast cells to propagate an immune response. Cytokines can induce various responses in the recipient cell. Cytokines can include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines can promote an inflammatory response. Examples of homeostatic cytokines include, but are not limited to, IL-2, IL-4, IL-5, IL-7, IL-10, IL-12p40, IL-12p′70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, the term refers to a cell that was isolated and subsequently introduced to other cells or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from where it would be in natural cells, or is otherwise flanked by a different nucleic acid sequence than that found in nature.

The term “expression construct” or “expression cassette” refers to a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

The term “vector” or “construct” (sometimes referred to as a gene delivery system or gene transfer “vehicle”) refers to a macromolecule or complex of molecules comprising a polynucleotide to be delivered to a host cell, either in vitro or in vivo.

The term “plasmid,” a common type of a vector, is an extra-chromosomal DNA molecule separate from the chromosomal DNA that is capable of replicating independently of the chromosomal DNA. In certain cases, it is circular and double-stranded.

The term “origin of replication” (“ori”) or “replication origin” is a DNA sequence, e.g., in a lymphotrophic herpes virus, that when present in a plasmid in a cell is capable of maintaining linked sequences in the plasmid and/or a site at or near where DNA synthesis initiates. As an example, an ori for EBV (Ebstein-Barr virus) includes FR sequences (20 imperfect copies of a 30 bp repeat), and preferably DS sequences; however, other sites in EBV bind EBNA-1, e.g., Rep* sequences can substitute for DS as an origin of replication (Kirshmaier and Sugden, 1998). Thus, a replication origin of EBV includes FR, DS or Rep* sequences or any functionally equivalent sequences through nucleic acid modifications or synthetic combination derived therefrom. For example, methods of the present disclosure may also use genetically engineered replication origin of EBV, such as by insertion or mutation of individual elements.

The term “gene,” “polynucleotide,” “coding region,” “sequence,” “segment,” “fragment,” or “transgene” that “encodes” a particular protein, is a nucleic acid molecule that is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “control elements” refers collectively to promoter regions, polyadenylation signals, transcription termination sequences, upstream regulatory domains, origins of replication, internal ribosome entry sites (IRES), enhancers, splice junctions, and the like, which collectively provide for the replication, transcription, post-transcriptional processing, and translation of a coding sequence in a recipient cell. Not all of these control elements need be present so long as the selected coding sequence is capable of being replicated, transcribed, and translated in an appropriate host cell.

The term “promoter” is used herein in its ordinary sense to refer to a nucleotide region comprising a DNA regulatory sequence, wherein the regulatory sequence is derived from a gene that is capable of binding RNA polymerase and initiating transcription of a downstream (3′ direction) coding sequence. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

The term “enhancer” is meant a nucleic acid sequence that, when positioned proximate to a promoter, confers increased transcription activity relative to the transcription activity resulting from the promoter in the absence of the enhancer domain.

The term “operably linked” or co-expressed” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. “Operably linked” or “co-expressed” with reference to peptide and/or polypeptide molecules means that two or more peptide and/or polypeptide molecules are connected in such a way as to yield a single polypeptide chain, i.e., a fusion polypeptide, having at least one property of each peptide and/or polypeptide component of the fusion. The fusion polypeptide is preferably chimeric, i.e., composed of heterologous molecules.

The term “Homology” refers to the percent of identity between two polynucleotides or two polypeptides. The correspondence between one sequence and another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions that promote the formation of stable duplexes between homologous regions, followed by digestion with single strand-specific nuclease(s), and size determination of the digested fragments. Two DNA, or two polypeptide, sequences are “substantially homologous” to each other when at least about 80%, preferably at least about 90%, and most preferably at least about 95% of the nucleotides, or amino acids, respectively match over a defined length of the molecules, as determined using the methods above.

The term “cell” is herein used in its broadest sense in the art and refers to a living body that is a structural unit of tissue of a multicellular organism, is surrounded by a membrane structure that isolates it from the outside, has the capability of self-replicating, and has genetic information and a mechanism for expressing it. Cells used herein may be naturally-occurring cells or artificially modified cells (e.g., fusion cells, genetically modified cells, etc.).

The term “stem cell” refers herein to a cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPScs or iPS cells”.

An “embryonic stem (ES) cell” is an undifferentiated pluripotent cell which is obtained from an embryo in an early stage, such as the inner cell mass at the blastocyst stage, or produced by artificial means (e.g. nuclear transfer) and can give rise to any differentiated cell type in an embryo or an adult, including germ cells (e.g. sperm and eggs).

“Induced pluripotent stem cells (iPScs or iPS cells)” are cells generated by reprogramming a somatic cell by expressing or inducing expression of a combination of factors (herein referred to as reprogramming factors). iPS cells can be generated using fetal, postnatal, newborn, juvenile, or adult somatic cells. In certain embodiments, factors that can be used to reprogram somatic cells to pluripotent stem cells include, for example, Oct4 (sometimes referred to as Oct 3/4), Sox2, c-Myc, Klf4, Nanog, and Lin28. In some embodiments, somatic cells are reprogrammed by expressing at least two reprogramming factors, at least three reprogramming factors, at least four reprogramming factors, at least five reprogramming factors, at least six reprogramming factors, or at least seven reprogramming factors to reprogram a somatic cell to a pluripotent stem cell.

“Hematopoietic progenitor cells” or “hematopoietic precursor cells” refers to cells which are committed to a hematopoietic lineage but are capable of further hematopoietic differentiation and include hematopoietic stem cells, multipotential hematopoietic stem cells, common myeloid progenitors, megakaryocyte progenitors, erythrocyte progenitors, and lymphoid progenitors. Hematopoietic stem cells (HSCs) are multipotent stem cells that give rise to all the blood cell types including myeloid (monocytes and macrophages, granulocytes (neutrophils, basophils, eosinophils, and mast cells), erythrocytes, megakaryocytes/platelets, dendritic cells), and lymphoid lineages (T-cells, B-cells, NK-cells) (see e.g., Doulatov et al., 2012; Notta et al., 2015). A “multilymphoid progenitor” (MLP) is defined to describe any progenitor that gives rise to all lymphoid lineages (B, T, and NK cells), but that may or may not have other (myeloid) potentials (Doulatov et al., 2010) and is CD45RA.sup.+, /CD10.sup.+/CD7.sup.−. Any B, T, and NK progenitor can be referred to as an MLP. A “common myeloid progenitor” (CMP) refers to CD45RA.sup.−/CD135.sup.+/CD10.sup.−/CD7.sup.− cells that can give rise to granulocytes, monocytes, megakaryocytes and erythrocytes.

“Pluripotent stem cell” refers to a stem cell that has the potential to differentiate into all cells constituting one or more tissues or organs, or preferably, any of the three germ layers: endoderm (interior stomach lining, gastrointestinal tract, the lungs), mesoderm (muscle, bone, blood, urogenital), or ectoderm (epidermal tissues and nervous system).

The term “somatic cell” refers to any cell other than germ cells, such as an egg, a sperm, or the like, which does not directly transfer its DNA to the next generation. Typically, somatic cells have limited or no pluripotency. Somatic cells used herein may be naturally-occurring or genetically modified.

The term “Programming” is a process that alters the type of progeny a cell can produce. For example, a cell has been programmed when it has been altered so that it can form progeny of at least one new cell type, either in culture or in vivo, as compared to what it would have been able to form under the same conditions without programming. This means that after sufficient proliferation, a measurable proportion of progeny having phenotypic characteristics of the new cell type are observed, if essentially no such progeny could form before programming; alternatively, the proportion having characteristics of the new cell type is measurably more than before programming. This process includes differentiation, dedifferentiation and transdifferentiation.

The term “Differentiation” is the process by which a less specialized cell becomes a more specialized cell type. “Dedifferentiation” is a cellular process in which a partially or terminally differentiated cell reverts to an earlier developmental stage, such as pluripotency or multipotency. “Transdifferentiation” is a process of transforming one differentiated cell type into another differentiated cell type. Typically, transdifferentiation by programming occurs without the cells passing through an intermediate pluripotency stage—i.e., the cells are programmed directly from one differentiated cell type to another differentiated cell type. Under certain conditions, the proportion of progeny with characteristics of the new cell type may be at least about 1%, 5%, 25% or more in order of increasing preference.

The term “subject” or “subject in need thereof” refers to a mammal, preferably a human being, male or female at any age that is in need of a cell or tissue transplantation. Typically the subject is in need of cell or tissue transplantation (also referred to herein as recipient) due to a disorder or a pathological or undesired condition, state, or syndrome, or a physical, morphological or physiological abnormality which is amenable to treatment via cell or tissue transplantation.

The term “disruption” or “alteration” of a gene refers to the elimination or reduction of expression of one or more gene products encoded by the subject gene in a cell, compared to the level of expression of the gene product in the absence of the alteration. Exemplary gene products include mRNA and protein products encoded by the gene. Alteration in some cases is transient or reversible and in other cases is permanent. Alteration in some cases is of a functional or full length protein or mRNA, despite the fact that a truncated or non-functional product may be produced. In some embodiments herein, gene activity or function, as opposed to expression, is disrupted. Gene alteration is generally induced by artificial methods, i.e., by addition or introduction of a compound, molecule, complex, or composition, and/or by alteration of nucleic acid of or associated with the gene, such as at the DNA level. Exemplary methods for gene alteration include gene silencing, knockdown, knockout, and/or gene alteration techniques, such as gene editing. Examples include antisense technology, such as RNAi, siRNA, shRNA, and/or ribozymes, which generally result in transient reduction of expression, as well as gene editing techniques which result in targeted gene inactivation or alteration, e.g., by induction of breaks and/or homologous recombination. Examples include insertions, mutations, and deletions. The alterations typically result in the repression and/or complete absence of expression of a normal or “wild type” product encoded by the gene. Exemplary of such gene alterations are insertions, frameshift and missense mutations, deletions, knock-in, and knock-out of the gene or part of the gene, including deletions of the entire gene. Such alterations can occur in the coding region, e.g., in one or more exons, resulting in the inability to produce a full-length product, functional product, or any product, such as by insertion of a stop codon. Such alterations may also occur by alterations in the promoter or enhancer or other region affecting activation of transcription, so as to prevent transcription of the gene. Gene alterations include gene targeting, including targeted gene inactivation by homologous recombination.

The term “immune disorder,” “immune-related disorder,” or “immune-mediated disorder” refers to a disorder in which the immune response plays a key role in the development or progression of the disease. Immune-mediated disorders include autoimmune disorders, allograft rejection, graft versus host disease and inflammatory and allergic conditions.

The term “immune response” is a response of a cell of the immune system, such as a B cell, or a T cell, or innate immune cell to a stimulus. In one embodiment, the response is specific for a particular antigen (an “antigen-specific response”).

The term “antigen” is a molecule capable of being bound by an antibody or T-cell receptor. An antigen may generally be used to induce a humoral immune response and/or a cellular immune response leading to the production of B and/or T lymphocytes.

The terms “tumor-associated antigen,” “tumor antigen” and “cancer cell antigen” are used interchangeably herein. In each case, the terms refer to proteins, glycoproteins or carbohydrates that are specifically or preferentially expressed by cancer cells.

The term “epitope” is the site on an antigen recognized by an antibody as determined by the specificity of the amino acid sequence. Two antibodies are said to bind to the same epitope if each competitively inhibits (blocks) binding of the other to the antigen as measured in a competitive binding assay. Alternatively, two antibodies have the same epitope if most amino acid mutations in the antigen that reduce or eliminate binding of one antibody reduce or eliminate binding of the other. Two antibodies are said to have overlapping epitopes if each partially inhibits binding of the other to the antigen, and/or if some amino acid mutations that reduce or eliminate binding of one antibody reduce or eliminate binding of the other.

In one embodiment of the invention CAR-T are generated using means known in the art except for the addition of inducible cytokine and/or chemokine production. provides a method of reducing the number of target cells including the steps of (i.) contacting said target cells with an effective amount of an engineered cell having at least one chimeric antigen receptor polypeptide, for engineered cells having multiple chimeric antigen receptor polypeptides, each chimeric antigen receptor polypeptide is independent; and (ii.) optionally, assaying for the reduction in the number of said cells. The target cells include at least one cell surface antigen selected from the group consisting of GD2, GD3, ROR1, PSMA, PSCA (prostate stem cell antigen), MAGE A3, Glycolipid, glypican 3, F77, GD-2, WT1, CEA, HER-2/neu, MAGE-3, MAGE-4, MAGE-5, MAGE-6, alpha-fetoprotein, CA 19-9, CA 72-4, NY-ESO, FAP, ErbB, c-Met, MART-1, MUC1, MUC2, MUC3, MUC4, MUC5, CD30, MMG49 epitope, EGFRvIII, CD33, CD123, CLL-1, immunoglobin kappa and lambda, CD38, CD52, CD47, CD200, CD70, CD19, CD20, CD22, CD38, BCMA, CS1, NKG2D receptor, April receptor, BAFF receptor, TACI, CD3, CD4, CD8, CD5, CD7, CD2, and CD138. The target antigens can also include viral or fungal antigens, such as E6 and E7 from the human papillomavirus (HPV) or EBV (Epstein Ban virus) antigens. In another embodiment, the present disclosure provides methods for treating B-cell lymphoma, T-cell lymphoma, multiple myeloma, chronic myeloid leukemia, acute myeloma leukemia, myelodysplastic syndromes, chronic myeloproliferative neoplasms, B-cell acute lymphoblastic leukemia (B-ALL), and cell proliferative diseases by administering any of the engineered cells described above to a patient in need thereof. In another embodiment, the present disclosure provides a method of treating an autoimmune disease, said method including administering an engineered cell according to claim 1 to a patient in need thereof; wherein said autoimmune disease comprises systemic lupus erythematosus (SLE), multiple sclerosis (MS), Inflammatory bowel disease (IBD), Rheumatoid arthritis, Sjogren syndrome, dermatomyosities, autoimmune hemolytic anemia, Neuromyelitis optica (NMO), NMO Spectrum Disorder (NMOSD), idiopathic thrombocytopenic purpura (ITP), antineutorphil cytoplasmic autoantibodies (ANCAs) associated with systemic autoimmune small vessel vasculitis syndromes or microscopic polyangiitis (MPA), granulomatosis with polyangiitis (GPA, Wegener's granulomatosis), or eosinophilic granulomatosis with polyangiitis (EGPA, Churg-Strauss syndrome) and TTP (thrombotic thrombocytopenic purpura). The present disclosure provides chimeric antigen receptors (CARS) targeting non-hematologic malignancies, compositions and methods of use thereof. In one embodiment, the present disclosure provides an engineered cell having a first chimeric antigen receptor polypeptide including a first antigen recognition domain, a first signal peptide, a first hinge region, a first transmembrane domain, a first co-stimulatory domain, and a first signaling domain; and a second chimeric antigen receptor polypeptide including a second antigen recognition domain, a second signal peptide, a second hinge region, a second transmembrane domain, a second co-stimulatory domain, and a second signaling domain; wherein the first antigen recognition domain is different than the second antigen recognition domain. In another embodiment, the present disclosure provides an engineered polypeptide including a chimeric antigen receptor and an enhancer (s). In a further embodiment, an enhancer can be selected from at least one of the group including, but not limited, IL-2, IL-7, IL-12, IL-15, IL-15/IL-15sush, IL-15/IL-15sushi anchor, IL-15/IL-15RA, IL-18, IL-21, IL-21 anchor, PD-1, PD-L1, CSF1R, CTAL-4, TIM-3, cytoplasmic domain of IL-15 receptor alpha, 4-1BBL, IL-21, IL-21 anchor and TGFR beta, receptors. In some embodiments, CAR having an antigen recognition domain (s) is part of an expression cassette. In a preferred embodiment, the expressing gene or the cassette may include an accessory gene or a tag or a part thereof. The accessory gene may be an inducible suicide gene or a part thereof, including, but not limited to, caspase 9 gene. The “suicide gene” ablation approach improves safety of the gene therapy and kills cells only when activated by a specific compound or a molecule. In some embodiments, the epitope tag is a c-myc tag, CD52, streptavidin-binding peptide (SBP), truncated EGFR gene (EGFRt) or a part or a combination thereof. In some embodiments, CAR cells can be ablated by administrating an anti-CD52 monoclonal antibody (CAMPATH) to a subject. In another embodiment, the present disclosure provides methods for treating soft tissue tumors, carcinoma, sarcomas, leukemia, and cell proliferative diseases by administering any of the engineered cells described above to a patient in need thereof.

In one embodiment of the invention, CAR-T cells are used together with immunization to tumors of the same type the patient is suffering from is provided prior to cytotoxic, or immunogenic cell death induction of the tumor. Immunization of the patient may be performed using known means in the art, using suitable adjuvants. Assessment of immunity is performed by quantifying reactivity of T cells or B cells in response to protein antigens or derivatives thereof, derivatives including peptide antigens or other antigenic epitopes. Responses may be assessed in terms of proliferative responses, cytokine release, antibody responses, or generation of cytotoxic T cells. Methods of assessing said responses are well known in the art. In a preferred embodiment, antibody responses are assessed to a panel of tumor associated proteins subsequent to immunization of patient. Antibody responses are utilized to guide which peptides will be utilized for prior immunization. For example, if a patient is immunized with tumor antigen on a weekly basis, the subsequent assessment of antibody responses is performed at approximately 1-3 months after initiation of immunization. Protocols for immunization include weekly, biweekly, or monthly. Assessment of antibody responses is performed utilizing standard enzyme linked immunosorbent (ELISA) assay. Assessment of antibodies is performed, in one embodiment of the invention, against proteins associated with tumor.

In one embodiment of the invention, immunity to a polyvalent tumor vaccine is induced utilizing a vaccine such as CanVaxin, or other polyvalent vaccine mixtures. This is used in synergy with CAR-T administration. Numerous tumor antigens can be utilized to amplify the immune response selectively, these can be chosen from known groups of tumor antigens such as ERG, WT1, ALS, BCR-ABL, Ras-mutant, MUC1, ETV6-AML, LMP2, p53 non-mutant, MYO-N, surviving, androgen receptor, RhoC, cyclin B1, EGFRvIII, EphA2, B cell or T cell idiotype, ML-IAP, BORIS, hTERT, PLAC1, HPV E6, HPV E7, OY-TES1, Her2/neu, PAX3, NY-BR-1, p53 mutant, MAGE A3, EpCAM, polysialic Acid, AFP, PAX5, NY-ESO1, sperm protein 17, GD3, Fucosyl GM1, mesothelin, PSMA, GD2, MAGE A1, sLe(x), HMWMAA, CYP1B1, sperm fibrous sheath protein, B7H3, TRP-2, AKAP-4, XAGE 1, CEA, Tn, GloboH, SSX2, RGS5, SART3, gp100, MelanA/MART1, Tyrosinase, GM3 ganglioside, Proteinase 3 (PR1), Page4, STn, Carbonic anhydrase IX, PSCA, Legumain, MAD-CT-1 (protamin2), PSA, Tie 2, MAD-CT2, PAP, PDGFR-beta, NA17, VEGFR2, FAP, LCK, Fos-related antigen, LCK, FAP.

Combination of polyvalent vaccines with other cellular therapies as the initial poly-immunogenic composition is envisioned within the context of the invention. In one embodiment cellular lysates of tumor cells, or tumor stem cells are loaded into dendritic cells. In one embodiment the invention provides a means of generating a population of cells with tumoricidal ability that are polyvalently reactive, to which focus is added by subsequent peptide specific vaccination. The generation of cytotoxic lymphocytes may be performed, in one embodiment by extracted 50 ml of peripheral blood from a cancer patient and peripheral blood monoclear cells (PBMC) are isolated using the Ficoll Method. PBMC are subsequently resuspended in 10 ml AIM-V media and allowed to adhere onto a plastic surface for 2-4 hours. The adherent cells are then cultured at 37° C. in AIM-V media supplemented with 1,000 U/mL granulocyte-monocyte colony-stimulating factor and 500 U/mL IL-4 after non-adherent cells are removed by gentle washing in Hanks Buffered Saline Solution (HBSS). Half of the volume of the GM-CSF and IL-4 supplemented media is changed every other day. Immature DCs are harvested on day 7. In one embodiment said generated DC are used to stimulate T cell and NK cell tumoricidal activity by pulsing with autologous tumor lysate. Specifically, generated DC may be further purified from culture through use of flow cytometry sorting or magnetic activated cell sorting (MACS), or may be utilized as a semi-pure population. DC pulsed with tumor lysate may be added into said patient in need of therapy with the concept of stimulating NK and T cell activity in vivo, or in another embodiment may be incubated in vitro with a population of cells containing T cells and/or NK cells. In one embodiment DC are exposed to agents capable of stimulating maturation in vitro and rendering them resistant to tumor derived inhibitory compounds such as arginase byproducts. Specific means of stimulating in vitro maturation include culturing DC or DC containing populations with a toll like receptor agonist. Another means of achieving DC maturation involves exposure of DC to TNF-alpha at a concentration of approximately 20 ng/mL. In order to activate T cells and/or NK cells in vitro, cells are cultured in media containing approximately 1000 IU/ml of interferon gamma. Incubation with interferon gamma may be performed for the period of 2 hours to the period of 7 days. Preferably, incubation is performed for approximately 24 hours, after which T cells and/or NK cells are stimulated via the CD3 and CD28 receptors. One means of accomplishing this is by addition of antibodies capable of activating these receptors. In one embodiment approximately, 2 ug/ml of anti-CD3 antibody is added, together with approximately 1 ug/ml anti-CD28. In order to promote survival of T cells and NK cells, was well as to stimulate proliferation, a T cell/NK mitogen may be used. In one embodiment the cytokine IL-2 is utilized. Specific concentrations of IL-2 useful for the practice of the invention are approximately 500 u/mL IL-2. Media containing IL-2 and antibodies may be changed every 48 hours for approximately 8-14 days. In one particular embodiment DC are included to said T cells and/or NK cells in order to endow cytotoxic activity towards tumor cells. In a particular embodiment, inhibitors of caspases are added in the culture so as to reduce rate of apoptosis of T cells and/or NK cells. Generated cells can be administered to a subject intradermally, intramuscularly, subcutaneously, intraperitoneally, intraarterially, intravenously (including a method performed by an indwelling catheter), intratumorally, or into an afferent lymph vessel. The immune response of the patient treated with these cytotoxic cells is assessed utilizing a variety of antigens found in tumor cells. When cytotoxic or antibody, or antibody associated with complement fixation are recognized to be upregulated in the cancer patient, subsequent immunizations are performed utilizing peptides to induce a focusing of the immune response.

In another embodiment DC are generated from leukocytes of patients by leukopheresis. Numerous means of leukopheresis are known in the art. In one example, a Frenius Device (Fresenius Com.Tec) is utilized with the use of the MNC program, at approximately 1500 rpm, and with a P1Y kit. The plasma pump flow rates are adjusted to approximately 50 mL/min. Various anticoagulants may be used, for example ACD-A. The Inlet/ACD Ratio may be ranged from approximately 10:1 to 16:1. In one embodiment approximately 150 mL of blood is processed. The leukopheresis product is subsequently used for initiation of dendritic cell culture. In order to generates a peripheral blood mononuclear cells from leukopheresis product, mononuclear cells are isolated by the Ficoll-Hypaque density gradient centrifugation. Monocytes are then enriched by the Percoll hyperosmotic density gradient centrifugation followed by two hours of adherence to the plate culture. Cells are then centrifuged at 500 g to separate the different cell populations. Adherent monocytes are cultured for 7 days in 6-well plates at 2×106 cells/mL RMPI medium with 1% penicillin/streptomycin, 2 mM L-glutamine, 10% of autologous, 50 ng/mL GM-CSF and 30 ng/mL IL-4. On day 6 immature dendritic cells are pulsed with tumor antigen. Pulsing may be performed by incubation of lysates with dendritic cells, or may be generated by fusion of immature dendritic cells with tumor cells. Means of generating hybridomas or cellular fusion products are known in the art and include electrical pulse mediated fusion, or stimulation of cellular fusion by treatment with polyethelyne glycol. On day 7, the immature DCs are then induced to differentiate into mature DCs by culturing for 48 hours with 30 ng/mL interferon gamma (IFN-γ). During the course of generating DC for clinical purposes, microbiologic monitoring tests are performed at the beginning of the culture, on the fifth day and at the time of cell freezing for further use or prior to release of the dendritic cells. Administration of tumor pulsed dendritic cells is utilized as a polyvalent vaccine, whereas subsequent to administration antibody or t cell responses are assessed for induction of antigen specificity, peptides corresponding to immune response stimulated are used for further immunization to focus the immune response.

In some embodiments, culture of the immune effectors cells is performed after extracting from a patient that has been immunized with a polyvalent antigenic preparation. Specifically separating the cell population and cell sub-population containing a T cell can be performed, for example, by fractionation of a mononuclear cell fraction by density gradient centrifugation, or a separation means using the surface marker of the T cell as an index. Subsequently, isolation based on surface markers may be performed. Examples of the surface marker include CD3, CD8 and CD4, and separation methods depending on these surface markers are known in the art. For example, the step can be performed by mixing a carrier such as beads or a culturing container on which an anti-CD8 antibody has been immobilized, with a cell population containing a T cell, and recovering a CD8-positive T cell bound to the carrier. As the beads on which an anti-CD8 antibody has been immobilized, for example, CD8 MicroBeads), Dynabeads M450 CD8, and Eligix anti-CD8 mAb coated nickel particles can be suitably used. This is also the same as in implementation using CD4 as an index and, for example, CD4 MicroBeads, Dynabeads M-450 CD4 can also be used. In some embodiments of the invention, T regulatory cells are depleted before initiation of the culture. Depletion of T regulatory cells may be performed by negative selection by removing cells that express makers such as neuropilin, CD25, CD4, CTLA4, and membrane bound TGF-beta. Experimentation by one of skill in the art may be performed with different culture conditions in order to generate effector lymphocytes, or cytotoxic cells, that possess both maximal activity in terms of tumor killing, as well as migration to the site of the tumor. For example, the step of culturing the cell population and cell sub-population containing a T cell can be performed by selecting suitable known culturing conditions depending on the cell population. In addition, in the step of stimulating the cell population, known proteins and chemical ingredients, etc., may be added to the medium to perform culturing. For example, cytokines, chemokines or other ingredients may be added to the medium. Herein, the cytokine is not particularly limited as far as it can act on the T cell, and examples thereof include IL-2, IFN-.gamma., transforming growth factor (TGF)-.beta., IL-15, IL-7, IFN-.alpha., IL-12, CD40L, and IL-27. From the viewpoint of enhancing cellular immunity, particularly suitably, IL-2, IFN-.gamma., or IL-12 is used and, from the viewpoint of improvement in survival of a transferred T cell in vivo, IL-7, IL-15 or IL-21 is suitably used. In addition, the chemokine is not particularly limited as far as it acts on the T cell and exhibits migration activity, and examples thereof include RANTES, CCL21, MIP1.alpha., MIP1.beta., CCL19, CXCL12, IP-10 and MIG. The stimulation of the cell population can be performed by the presence of a ligand for a molecule present on the surface of the T cell, for example, CD3, CD28, or CD44 and/or an antibody to the molecule. Further, the cell population can be stimulated by contacting with other lymphocytes such as antigen presenting cells (dendritic cell) presenting a target peptide such as a peptide derived from a cancer antigen on the surface of a cell. In addition to assessing cytotoxicity and migration as end points, it is within the scope of the current invention to optimize the cellular product based on other means of assessing T cell activity, for example, the function enhancement of the T cell in the method of the present invention can be assessed at a plurality of time points before and after each step using a cytokine assay, an antigen-specific cell assay (tetramer assay), a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. In vivo assessment of the efficacy of the generated cells using the invention may be assessed in a living body before first administration of the T cell with enhanced function of the present invention, or at various time points after initiation of treatment, using an antigen-specific cell assay, a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. Further, an immune response can be assessed by a weight, diameter or malignant degree of a tumor possessed by a living body, or the survival rate or survival term of a subject or group of subjects. Said cells can be expanded in the presence of specific antigens associated with tumors and subsequently injected into the patient in need of treatment. Expansion with specific antigens includes coculture with proteins selected from a group comprising of: a) ROBO; b) VEGF-R2; c) FGF-R; d) CD105; e) TEM-1; and f) survivin. Other tumor specific or semi-specific antigens are known in the art that may be used.

Within the context of the invention, teachings are provided to amplify an antigen specific immune response following immunization with a polyvalent vaccine, in which the antigenic epitopes are used for immunization together with adjuvants such as toll like receptors (TLRs). These molecules are type 1 membrane receptors that are expressed on hematopoietic and non-hematopoietic cells. At least 11 members have been identified in the TLR family. These receptors are characterized by their capacity to recognize pathogen-associated molecular patterns (PAMP) expressed by pathogenic organisms. It has been found that triggering of TLR elicits profound inflammatory responses through enhanced cytokine production, chemokine receptor expression (CCR2, CCR5 and CCR7), and costimulatory molecule expression. As such, these receptors in the innate immune systems exert control over the polarity of the ensuing acquired immune response. Among the TLRs, TLR9 has been extensively investigated for its functions in immune responses. Stimulation of the TLR9 receptor directs antigen-presenting cells (APCs) towards priming potent, T.sub.H1-dominated T-cell responses, by increasing the production of pro-inflammatory cytokines and the presentation of co-stimulatory molecules to T cells. CpG oligonucleotides, ligands for TLR9, were found to be a class of potent immunostimulatory factors. CpG therapy has been tested against a wide variety of tumor models in mice, and has consistently been shown to promote tumor inhibition or regression.

In some embodiments of the invention, specific antigens are immunized following polyvalent immunization, said specific antigens administered in the form of DNA vaccines. The nucleic acid compositions, including the DNA vaccine compositions, may further comprise a pharmaceutically acceptable excipient. Examples of suitable pharmaceutically acceptable excipients for nucleic acid compositions, including DNA vaccine compositions, are well known to those skilled in the art and include sugars, etc. Such excipients may be aqueous or non aqueous solutions, suspensions, and emulsions. Examples of non-aqueous excipients include propylene glycol, polyethylene glycol, vegetable oils such as olive oil, and injectable organic esters such as ethyl oleate. Examples of aqueous excipient include water, alcoholic/aqueous solutions, emulsions or suspensions, including saline and buffered media. Suitable excipients also include agents that assist in cellular uptake of the polynucleotide molecule. Examples of such agents are (i) chemicals that modify cellular permeability, such as bupivacaine, (ii) liposomes or viral particles for encapsulation of the polynucleotide, or (iii) cationic lipids or silica, gold, or tungsten microparticles which associate themselves with the polynucleotides. Anionic and neutral liposomes are well-known in the art (see, e.g., Liposomes: A Practical Approach, RPC New Ed, IRL press (1990), for a detailed description of methods for making liposomes) and are useful for delivering a large range of products, including polynucleotides. Cationic lipids are also known in the art and are commonly used for gene delivery. Such lipids include Lipofectin™ also known as DOTMA (N-[I-(2,3-dioleyloxy) propyls N,N, N-trimethylammonium chloride), DOTAP (1,2-bis (oleyloxy)-3 (trimethylammonio) propane), DDAB (dimethyldioctadecyl-ammonium bromide), DOGS (dioctadecylamidologlycyl spermine) and cholesterol derivatives such as DCChol (3 beta-(N-(N′,N′-dimethyl aminomethane)-carbamoyl) cholesterol). A description of these cationic lipids can be found in EP 187,702, WO 90/11092, U.S. Pat. No. 5,283,185, WO 91/15501, WO 95/26356, and U.S. Pat. No. 5,527,928. A particular useful cationic lipid formulation that may be used with the nucleic vaccine provided by the disclosure is VAXFECTIN, which is a commixture of a cationic lipid (GAP-DMORIE) and a neutral phospholipid (DPyPE) which, when combined in an aqueous vehicle, self-assemble to form liposomes. Cationic lipids for gene delivery are preferably used in association with a neutral lipid such as DOPE (dioleyl phosphatidylethanolamine), as described in WO 90/11092 as an example. In addition, a DNA vaccine can also be formulated with a nonionic block copolymer such as CRL1005. Other immunization means include prime boost regiments [34]. The polypeptide and nucleic acid compositions can be administered to an animal, including human, by a number of methods known in the art. Examples of suitable methods include: (1) intramuscular, intradermal, intraepidermal, intravenous, intraarterial, subcutaneous, or intraperitoneal administration, (2) oral administration, and (3) topical application (such as ocular, intranasal, and intravaginal application). One particular method of intradermal or intraepidermal administration of a nucleic acid vaccine composition that may be used is gene gun delivery using the Particle Mediated Epidermal Delivery (PMED™) vaccine delivery device marketed by PowderMed [35]. PMED is a needle-free method of administering vaccines to animals or humans. The PMED system involves the precipitation of DNA onto microscopic gold particles that are then propelled by helium gas into the epidermis. The DNA-coated gold particles are delivered to the APCs and keratinocytes of the epidermis, and once inside the nuclei of these cells, the DNA elutes off the gold and becomes transcriptionally active, producing encoded protein. This protein is then presented by the APCs to the lymphocytes to induce a T-cell-mediated immune response. Another particular method for intramuscular administration of a nucleic acid vaccine provided by the present disclosure is electroporation. Electroporation uses controlled electrical pulses to create temporary pores in the cell membrane, which facilitates cellular uptake of the nucleic acid vaccine injected into the muscle. Where a CpG is used in combination with a nucleic acid vaccine, it is preferred that the CpG and nucleic acid vaccine are co-formulated in one formulation and the formulation is administered intramuscularly by electroporation. A helper T cell and cytotoxic T cell stimulatory polypeptide can be introduced into a mammalian host, including humans, linked to its own carrier or as a homopolymer or heteropolymer of active polypeptide units. Such a polymer can elicit increase immunological reaction and, where different polypeptides are used to make up the polymer, the additional ability to induce antibodies and/or T cells that react with different antigenic determinants of the tumor. Useful carriers known in the art include, for example, thyroglobulin, albumins such as human serum albumin, tetanus toxoid, polyamino acids such as poly(D-lysine:D-glutamic acid), influenza polypeptide, and the like. Adjuvants such as incomplete Freunds adjuvant, GM-CSF, aluminum phosphate, CpG containing DNA, inulin, Poly (IC), aluminum hydroxide, alum, or montanide can also be used in the administration of an helper T cell and cytotoxic T cell stimulatory polypeptide.

Subsequent to augmentation of lymphocyte numbers specific for killing of the tumor, modification of the tumor microenvironment may be performed. In one embodiment, macrophage modulators are used.

Macrophages can play a tumor inhibitory, as well as a tumor stimulatory role. Initial studies supported the role of macrophages in mediating antibody dependent cellular cytotoxicity in tumors, and thus being associated with potentiation of antitumor immune responses. Macrophages also possess the ability to directly recognize tumors by virtue of tumor expressed “eat-me” signals, which include the stress associated protein calreticulin, which binds to the low-density lipoprotein receptor-related protein (LRP) on macrophages to induce phagocytosis. Tumors protect themselves by expression of CD47, which binds to macrophage SIRP-1 and transduces an inhibitory signal. Blockade of CD47 using antibodies results in remission of cancers mediated by macrophage activation. Thus on the one hand, macrophages play an important role in induction of antitumor immunity. This can also be exemplified by some studies, involving administration of GM-CSF in order to augment macrophage numbers and activity in cancer patients.

Unfortunately, there is also evidence that macrophages support tumor growth. Studies in the osteopetrotic mice strain, which lacks mature macrophages, demonstrate that tumors actually grow slower in animals deficient in macrophages. Several other animal models have elegantly demonstrated that macrophages contribute to tumor growth, in part through stimulating on the angiogenic switch. Numerous tumor biopsy studies have shown that there is a negative correlation between macrophage infiltration into tumors and patient survival.

The duality of macrophages in growth of tumors may be seen in studies of “inverse hormesis” in which low concentrations of antibodies targeting the tumor specific marker sialic acid N-glycolyl-neuraminic acid (Neu5Gc) actually leads to enhanced tumor growth in a macrophage dependent manner.

The importance of macrophages in clinical implementation of cancer therapeutics can be seen from results of a double blind clinical trials in metastatic colorectal cancer patients where cetuximab (anti-epidermal growth factor receptor (EGFR) monoclonal antibody (mAb)) was added to a protocol comprising of bevacizumab and chemotherapy. The addition of cetuximab actually resulted in decreased survival. In a study examining whether monocyte conversion to M2 angiogenic macrophages was responsible, investigators observed that CD163-positive M2 macrophages where found in high concentrations intratumorally in patients with colorectal carcinomas. These M2 cells expressed abundant levels of Fc-gamma receptors (FcγR) and PD-L1. Additionally, consistent with the M2 phenotype the cells generated large amounts of the immunosuppressive molecule IL-10 and the angiogenic mediator VEGF. When M2 cells were cultured with EGFR-positive tumor cells loaded with low concentrations of cetuximab, further augmentation of IL-10 and VEGF production was observed. These data suggest that under certain contexts, tumors manipulate macrophages to take on the M2 phenotype, and this subsequently leads to enhanced tumor progressing factors when tumor cells are bound by antibodies.

Manipulation of macrophages to inhibit M2 and shift to M1 phenotype may be performed using a variety of means. One theme that seems unifying is the ability of toll like receptor (TLR) agonists to influence this. In addition to cytokine differences, macrophages capable of killing tumor cells are usually known to express low levels of the inhibitory Fc gamma receptor IIb, whereas tumor promoting macrophages have high levels of this receptor. Furthermore, tumor associated cytokines such as IL-4 and IL-10 are known to induce upregulation of the Fc gamma receptor which is immune suppressive.

In one study, the effect of the TLR7/8 agonist R-848 was assessed on monocytes derived from human peripheral blood. It was found that 12 hour exposure of R-848 increased FcgammaR-mediated cytokine production and antibody-dependent cellular cytotoxicity by monocytes. Furthermore, upregulation of the ADCC associated receptors FcgammaRI, FcgammaRIIa, and the common gamma-subunit was observed. However treatment with R-848 led to profound downregulation of the inhibitory FcgammaRIIb molecule. These data support ability to modify therapeutic activity of macrophages by manipulation of TLR signaling pathways. Other TLRs have been found to suppress inhibitory receptors on macrophages. For example, in another study it was observed that exposing monocytes to TLR4 agonists leads to suppression of the FcγRIIb macrophage inhibitory protein by MARCH3 mediated ubiquitination.

In one embodiment administration of ImmunoMax is performed systemically, and/or locally, which is an injectable polysaccharide purified from potato sprouts and approved as pharmaceutical in the Russian Federation (registration P No.001919/02-2002) and 5 other countries of Commonwealth of Independent States (formerly the USSR) and has been evaluated in a wide range of medical situations. In accordance with the formal “Instruction of Medical Use”, one medical indication for Immunomax® is the stimulation of immune defense during the treatment of different infectious diseases (http://www.gepon.ru/immax_intro.htm). Studies have shown that Immunomax® induces immune mediated killing of cancer cells in a TLR4 dependent manner. In one embodiment of the invention, ImmunoMax is utilized to induce an M2 to M1 shift, thus reducing macrophage derived immune suppressants and augmenting production of immune stimulatory cytokines such as IL-12 and TNF-alpha. In some embodiments of the invention, other agents may be used to modulate M2 to M1 transition of tumor associated macrophages including RRx-001, the bee venom derived peptide melittin, CpG DNA, metformin, Chinese medicine derivative puerarin, rhubarb derivative emodin, dietary supplement chlorogenic acid, propranolol, poly ICLC, BCG, Agaricus blazei Murill mushroom extract, endotoxin, olive skin derivative maslinic acid, intravenous immunoglobulin, phosphotidylserine targeting antibodies, dimethyl sulfoxide, surfactant protein A, Zoledronic acid, and bacteriophages.

Prior to induction of immunogenic cell death, antigen presenting cells are administered within the current invention, one of the most potent antigen presenting cells is the dendritic cell.

Dendritic cells (DC) possess unique morphology similar to neuronal dendrites and were originally identified based on their ability to stimulate the adaptive immune system. Of importance to the field of tumor immunotherapy, dendritic cells appear to be the only cell in the body capable of activating naïve T cells. The concept of dendritic cells instructing naïve T cells to differentiate into effector or memory cells is fundamental because it places the dendritic cell as the most powerful antigen presenting cell. This implies that for immunotherapeutic purposes dendritic cells do not necessarily need to be administered at high numbers in patients.

The possibility of utilizing DC to stimulate immunity was made into reality in animal studies that took advantage of the ability of immature DC to potently phagocytose various antigens. If the antigens possessed DAMPs, or if DAMPs were present in the environment, the DC would mature and present the antigens, resulting in stimulation of potent T cell immunity. Accordingly, in the initial studies, immature DC were incubated with various antigens, subsequent to which a maturation signal (replicating natural DAMPs) was applied and the DC were injected into animals. Thus DC were utilized as a type of “cellular adjuvant”. Indeed, it was discovered that the classical adjuvants such as Fruend's Adjuvant actually contained a high concentration of DAMPs, which resulted in the stimulation of local DC at vaccination site in vivo.

1. A method of treating cancer comprising the steps of: a) obtaining a T cell population possessing a T cell receptor (TCR) capable of recognizing one or more tumor antigens; b) transfecting said T cell population with a construct in a manner such that activation of T cell receptor or signaling components associated with said T cell receptor results in stimulation of chemokine production from said T cell; and c) administering said T cell to a patient suffering from cancer.

2. The method of claim 1, wherein said T cell population is selected from the group consisting of: a) CD4 T cells; b) CD8 T cells; c) NKT cells; d) gamma delta T cells and e) innate lymphoid like cells.

3. The method of claim 1, wherein said CD4 cell is selected from the group consisting of: a) Th1; b) Th2; c) Th3; d) Th9; and e) Th17.

4. The method of claim 1, wherein said T cell population is capable of inducing chemoattraction of dendritic cells.

5. The method of claim 4, wherein said dendritic cell is capable of inducing antigen presentation.

6. The method of claim 5, wherein said antigen presentation involves expression of MHC I and/or MHCII on the surface of a dendritic cell, in which said MHC I and/or MHC II contains antigenic peptides.

7. The method of claim 5, wherein said antigen presentation involves expression of costimulatory molecules.

8. The method of claim 7, wherein said costimulatory molecule is selected from the group consisting of: CD40, CD80, CD86, interleukin-12, interleukin-12, ICAM-1, LFA-1, ICOS ligand, and OX40 ligand.

9. The method of claim 1, wherein said cancer antigen is selected from the group consisting of: Antigens known to be found on cancer, and useful for the practice of the invention include: pidermal growth factor receptor (EGFR, EGFR1, ErbB-1, HER1). ErbB-2 (HER2/neu), ErbB-3/HER3, ErbB-4/HER4, EGFR ligand family; insulin-like growth factor receptor (IGFR) family, IGF-binding proteins (IGFBPs), IGFR ligand family (IGF-1R); platelet derived growth factor receptor (PDGFR) family, PDGFR ligand family; fibroblast growth factor receptor (FGFR) family, FGFR ligand family, vascular endothelial growth factor receptor (VEGFR) family, VEGF family; HGF receptor family: TRK receptor family; ephrin (EPH) receptor family: AXL receptor family; leukocyte tyrosine kinase (LTK) receptor family; TIE receptor family, angiopoietin 1, 2; receptor tyrosine kinase-like orphan receptor (ROR) receptor family; discoidin domain receptor (DDR) family; RET receptor family; KLG receptor family; RYK receptor family; MuSK receptor family; Transforming growth factor alpha (TGF-.alpha.), TGF-.alpha. receptor; Transforming growth factor-beta (TGF-.beta.), TGF-.beta. receptor; Interleukin .beta. receptor alpha2 chain (IL13Ralpha2), Interleukin-6 (IL-6), IL-6 receptor, interleukin-4, IL-4 receptor, Cytokine receptors, Class I (hematopoietin family) and Class II (interferon/1L-10 family) receptors, tumor necrosis factor (TNF) family, TNF-.alpha., tumor necrosis factor (TNF) receptor superfamily (TNTRSF), death receptor family, TRAIL-receptor; cancer-testis (CT) antigens, lineage-specific antigens, differentiation antigens, alpha-actinin-4, ARTC1, breakpoint cluster region-Abelson (Bcr-abl) fusion products, B-RAF, caspase-5 (CASP-5), caspase-8 (CASP-8), beta-catenin (CTNNB1), cell division cycle 27 (CDC27), cyclin-dependent kinase 4 (CDK4), CDKN2A, COA-1, dek-can fusion protein, EFTUD-2, Elongation factor 2 (ELF2), Ets variant gene 6/acute myeloid leukemia 1 gene ETS (ETC6-AML1) fusion protein, fibronectin (FN), GPNMB, low density lipid receptor/GDP-L fucose: beta-Dgalactose 2-alpha-Lfucosyltraosferase (LDLR/FUT) fusion protein, HLA-A2, MLA-A11, heat shock protein 70-2 mutated (HSP70-2M), KIAA0205, MART2, melanoma ubiquitous mutated 1, 2, 3 (MUM-1, 2, 3), prostatic acid phosphatase (PAP), neo-PAP, Myosin class 1, NFYC, OGT, OS-9, pml-RARalpha fusion protein, PRDX5, PTPRK, K-ras (KRAS2), N-ras (NRAS), HRAS, RBAF600, SIRT12, SNRPD1, SYT-SSX1 or -SSX2 fusion protein, Triosephosphate Isomerase, BAGE, BAGE-1, BAGE-2, 3, 4, 5, GAGE-1, 2, 3, 4, 5, 6, 7, 8, GnT-V (aberrant N-acetyl glucosaminyl transferase V, MGAT5), HERV-K MEL, KK-LC, KM-HN-1, LAGE, LAGE-1, CTL-recognized antigen on melanoma (CAMEL), MAGE-A1 (MAGE-1). MAGE-A2, MAGE-A3, MAGE-A4, MAGE-AS, MAGE-A6, MAGE-A8, MAGE-A9, MAGE-A10. MAGE-A11, MAGE-A12, MAGE-3, MAGE-B1, MAGE-B2, MAGE-B5. MAGE-B6, MAGE-C1, MAGE-C2, mucin 1 (MUC1), MART-1/Melan-A (MLANA), gp100, gp100/Pme117 (S1LV), tyrosinase (TYR), TRP-1, HAGE, NA-88, NY-ESO-1, NY-ESO-1/LAGE-2, SAGE, Sp17. SSX-1, 2, 3, 4, TRP2-1NT2, carcino-embryonic antigen (CEA), Kallikrein 4, mammaglobin-A, OA1, prostate specific antigen (PSA), prostate specific membrane antigen, TRP-1/, 75. TRP-2 adipophilin, interferon inducible protein absent in melanoma 2 (AIM-2). BING-4, CPSF, cyclin D1, epithelial cell adhesion molecule (Ep-CAM), EpbA3, fibroblast growth factor-5 (FGF-5), glycoprotein 250 (gp250intestinal carboxyl esterase (iCE), alpha-feto protein (AFP), M-CSF, mdm-2, MUCI, p53 (TP53), PBF, PRAME, PSMA, RAGE-1, RNF43, RU2AS, SOX10, STEAP1, survivin (BIRCS), human telomerase reverse transcriptase (hTERT), telomerase, Wilms' tumor gene (WT1), SYCP1, BRDT, SPANX, XAGE, ADAM2, PAGE-5, LIP1, CTAGE-1, CSAGE, MMA1, CAGE, BORIS, HOM-TES-85, AF15q14, HCA66I, LDHC, MORC, SGY-1, SPO11, TPX1, NY-SAR-35, FTHLI7, NXF2 TDRD1, TEX 15, FATE, TPTE, immunoglobulin idiotypes, Bence-Jones protein, estrogen receptors (ER), androgen receptors (AR), CD40, CD30, CD20, CD19, CD33, CD4, CD25, CD3, cancer antigen 72-4 (CA 72-4), cancer antigen 15-3 (CA 15-3), cancer antigen 27-29 (CA 27-29), cancer antigen 125 (CA 125), cancer antigen 19-9 (CA 19-9), beta-human chorionic gonadotropin, 1-2 microglobulin, squamous cell carcinoma antigen, neuron-specific enolase, heat shock protein gp96. GM2, sargramostim, CTLA-4, 707 alanine proline (707-AP), adenocarcinoma antigen recognized by T cells 4 (ART-4), carcinoembryogenic antigen peptide-1 (CAP-1), calcium-activated chloride channel-2 (CLCA2), cyclophilin B (Cyp-B), and human signet ring tumor-2 (HST-2).

10. The method of claim 1, wherein said T cells are transfected with a construct that upon activation induces production of CXCL10.

11. The method of claim 1, wherein said T cells are transfected with a construct that upon activation induces production of CXCL11.

12. The method of claim 1, wherein upon activation of said CAR said T cell produces a cytokine associated with induction of T cell mediated cancer immunity.

13. The method claim 12, wherein said cytokine associated with induction of said T cell mediated cancer immunity is interferon alpha.

14. The method claim 12, wherein said cytokine associated with induction of said T cell mediated cancer immunity is interferon beta.

15. The method claim 12, wherein said cytokine associated with induction of said T cell mediated cancer immunity is interferon gamma.

16. The method claim 12, wherein said cytokine associated with induction of said T cell mediated cancer immunity is interferon tau.

17. The method claim 12, wherein said cytokine associated with induction of said T cell mediated cancer immunity is interferon omega.

18. The method claim 12, wherein said cytokine associated with induction of said T cell mediated cancer immunity is interleukin-17.

19. The method claim 12, wherein said cytokine associated with induction of said T cell mediated cancer immunity is LIGHT.

20. The method claim 12, wherein said cytokine associated with induction of said T cell mediated cancer immunity is TRAIL.
